(19) United States
(12) Patent Application Publication
(10) Pub. No.: US 2026/0305979 A1
(43) Pub. Date: Oct. 8, 2026

(54) FEEDBACK-ENABLED MAGNETIC ASSISTANCE DEVICE FOR EYELID MANAGEMENT

(71) Applicant: Ching-Feng LIEN, Kaohsiung City (TW)

(72) Inventors: Ching-Feng LIEN, Kaohsiung City (TW); Bor-Shyh LIN, Tainan City (TW)

(21) Appl. No.: 19/405,706

(22) Filed: Dec. 2, 2025

(30) Foreign Application Priority Data
Apr. 7, 2025 (TW) 114113006

(51) Int. Cl. A61B 5/11; A61B 5/00

(52) U.S. Cl. CPC A61B 5/1103; A61B 5/6803

(57) ABSTRACT

A device for eyelid treatment has a wearable object, a magnetic element, a biosignal sensor, an electromagnetic actuator, a controller, and a release button. The wearable object is wearable on a user's face and the magnetic element is to be adhered to an affected eyelid. The biosignal sensor on the wearable object detects muscle activities in a periorbital area of the user and generates sensing data. The controller activates or deactivates the electromagnetic actuator based on the sensing data. When the electromagnetic actuator is activated to attract the magnetic element, the affected eyelid is allowed to open or close. When the release button triggered, vibration sensing signal exceeds the threshold, captured image suddenly changes beyond a certain level, or pressure from thin-film pressure sensors suddenly becomes unbalanced or decreases, the magnetic attraction between the electromagnetic actuator and the magnetic element is interrupted, thus preventing injuries to the user’s eyelid.

### CROSS REFERENCE TO RELATED APPLICATIONS

This non-provisional application claims the benefit under 35 U.S.C. §119(a) to Patent Application No. 114113006 filed in Taiwan on Apr. 7, 2025, which is hereby expressly incorporated by reference into the present application.

### BACKGROUND OF THE INVENTION

### 1. Field of the Invention

The present invention is a device for assisting the treatment of eyelids, particularly to a device for management of blepharoptosis and lagophthalmos.

### 2. Description of the Related Art

Facial nerve paralysis may cause symptoms of asymmetrical facial muscle tone, asymmetrical smile, inability to close the eyelids (lagophthalmos), etc. Without adequate management, complications resulting from lagophthalmos including keratitis, corneal ulcers, and even blindness may occur. Current treatments for lagophthalmos include metal implants, tarsal suturing, epicanthoplasty, medication, and supportive care. Some studies show that electrical stimulation of the facial nerve or orbicularis oculi may improve lagophthalmos.

The common causes of acquired blepharoptosis include age‐related aponeurotic dysfunction, birth trauma, traumatic injury, contact lens induced injury, myasthenia gravis, dysfunction of the third cranial nerve, and postocular surgery complications. Surgical intervention is the mainstay treatment for blepharoptosis. Although there are surgical treatments for blepharoptosis, they are often associated with a high rate of reoperations or postsurgical complications. Currently, there are few practical devices that can improve blepharoptosis and lagophthalmos.

### SUMMARY OF THE INVENTION

An objective of the present disclosure is to provide a feedback-enabled magnetic assistance device for eyelid management, particularly to a device for management of blepharoptosis and lagophthalmos.

The feedback-enabled magnetic assistance device for eyelid management comprises:

a wearable object configured to be worn on a user’s face;

a magnetic element adapted to be affixed on an affected eyelid of the user;

a biosignal sensor mounted on the wearable object, detecting muscle activities in a periorbital area of the user, and producing sensing data corresponding to the muscle activities;

an electromagnetic actuator mounted on the wearable object at a position corresponding to the magnetic element, wherein the electromagnetic actuator is controllable to be activated to generate a magnetic attraction between the magnetic element and the electromagnetic actuator, or alternatively is controllable to be deactivated to interrupt the magnetic attraction;

a controller electrically connected to the biosignal sensor and the electromagnetic actuator via a signal wire, and configured to activate or deactivate the electromagnetic actuator according to the sensing data acquired from the biosignal sensor; and

a release button electrically configured on the controller, wherein the controller deactivates the electromagnetic actuator while the release button is triggered.

The magnetic element such as an iron sheet is designed to be attached to an affected eyelid like the paralytic upper eyelid. After the biosignal sensor detects muscle activities of the healthy eyelid or other facial characteristics around the periorbital skin and accordingly outputs sensing data to the controller, the controller determines the eye closure and opening. The controller thus activates the electromagnetic actuator to attract the magnetic element, or deactivates the electromagnetic actuator to interrupt the magnetic attraction therebetween, thereby controlling the affected eyelid to achieve opening or closing actions.

Other objectives, advantages and novel features of the invention will become more apparent from the following detailed description when taken in conjunction with the accompanying drawings.

### BRIEF DESCRIPTION OF THE DRAWINGS

FIG. 1 is a circuit block diagram of a feedback-enabled magnetic assistance device for eyelid management according to one embodiment of the invention.

FIGS. 2 and 3 are perspective views of the magnetic assistance device according to different embodiments of the invention.

FIG. 4 is a schematic view showing the magnetic assistance device being worn on a user’s face.

FIG. 5 is a circuit block diagram of the magnetic assistance device according to another embodiment of the invention .

FIG. 6 is a perspective view showing the magnetic assistance device having a vibration sensor according to another embodiment of the invention.

FIG. 7 illustrates the magnetic assistance device with an auxiliary bar mounted on lower rims of a frame.

FIGS. 8A and 8B illustrate the magnetic assistance device having at least one camera according to other embodiments of the invention.

FIGS. 9A and 9B illustrate the magnetic assistance device having a pair of thin-film pressure sensors according to other embodiments of the invention.

FIG. 10 is a circuit block diagram for the magnetic assistance devices of FIGS. 8A-9B.

FIG. 11 the magnetic assistance device having two vibration sensors according to other embodiments of the invention.

### DETAILED DESCRIPTION OF THE INVENTION

Directional terms as used herein, for example, up, down, right, left, front, back, top, bottom are made only with reference to the figures as illustrated and are not intended to imply absolute orientation unless otherwise specified.

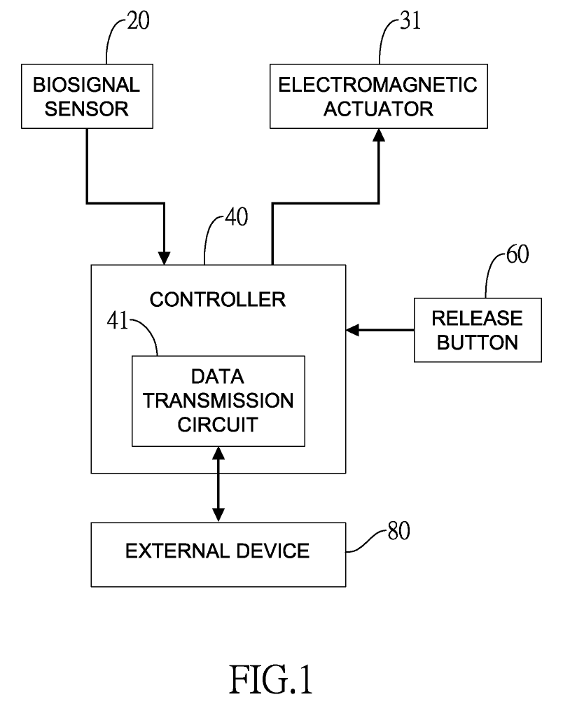

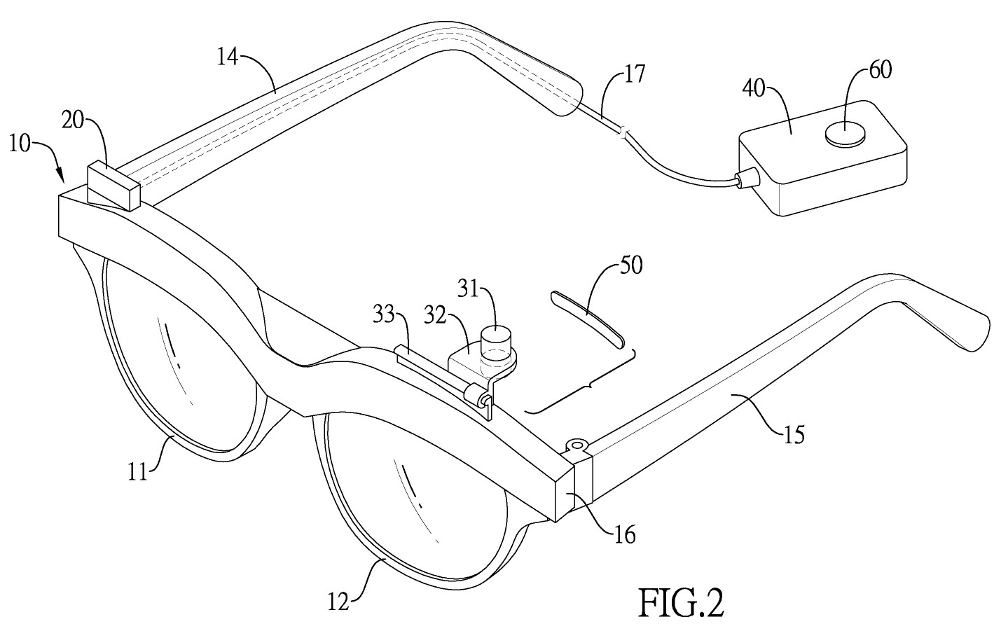

With reference to FIGS. 1 and 2, a feedback-enabled magnetic assistance device for eyelid management according to an embodiment of the present invention comprise a wearable object 10, a biosignal sensor 20, an electromagnetic actuator 31, a controller 40, a magnetic element 50 and a release button 60.

The wearable object 10 such as a glasses frame is configured to be worn on a user’s face. According to the embodiment, the wearable object 10 includes a first lens frame 11, a second lens frame 12 connected to the first lens frame 11 by a bridge (not shown), a first temple 14 hinged to the first lens frame 11, a second temple 15 hinged to the second lens frame 12, and an auxiliary bar 16 mounted on top rims of the first lens frame 11 and the second lens frame 12.

As an example in the following description, the first lens frame 11 is at the side corresponding to the user’s healthy eyelid (normal side) while the second lens frame 12 is at the contralateral side corresponding to the user’s paralytic eyelid (paralytic side). The paralytic eyelid hereinafter refers to a droopy eyelid (eyelid ptosis). The wearable object 10 can be manufactured entirely or partially using 3D printing technology, forming some chambers to accommodate the biosignal sensor 20, the electromagnetic actuator 31, etc. By embedding elements in the wearable objecting 10, the biosignal sensor 20 and the electromagnetic actuator 31 can be concealed within the wearable object 10 to prevent the user from attracting strange looks from others.

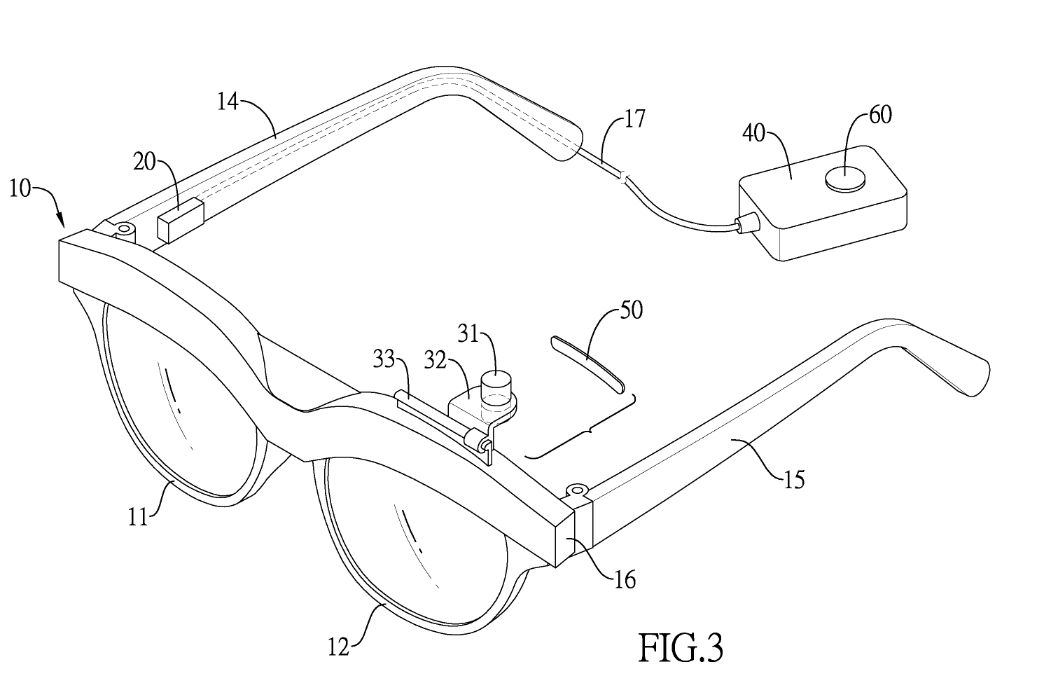

In the embodiment, the biosignal sensor 20 is configured to detect eye blinks or muscle activities near the periorbital area during eye closure or opening and accordingly output sensing data. The biosignal sensor 20 is preferably mounted on the wearable object 10 corresponding to the user’s healthy eyelid. The biosignal sensor 20 may be implemented by a camera or an optical sensor composed of a near-infrared emitter and a near-infrared receiver. As shown in FIG. 2, the biosignal sensor 20 is located on the auxiliary bar 16 above the first lens frame 11. Alternatively as shown in FIG. 3, the biosignal sensor 20 in another embodiment is mounted on an inner surface of the first temple 14 and near the first lens frame 11.

The biosignal sensor 20 may be located approximately an appropriate distance, such as one centimeter, away from the lateral canthus of the normal eyelid depending on the user’s condition. The near-infrared emitter periodically emits an infrared light toward the periorbital area such as the muscles behind the lateral canthus and detects changes in muscle contractions during eyes closure or opening. The near-infrared receiver may receive the infrared light reflected from the periorbital area, preferably muscles behind the lateral canthus, and accordingly produce a reflection signal as the sensing data.

The electromagnetic actuator 31 is directly or indirectly mounted on the wearable object 10 at the side corresponding to the paralytic eyelid, and may include a permanent magnet and an electromagnet. When the electromagnetic actuator 31 is activated, the electromagnetic actuator 31 is driven by an electrical current and continuously produces a magnetic force to attract the magnetic element 50. When the electromagnetic actuator 31 is deactivated, the electromagnetic actuator 31 stops generating the magnetic force, thus enabling the release of the magnetic element 50 from the electromagnetic actuator 31.

In the embodiment, the electromagnetic actuator 31 is indirectly mounted on the auxiliary bar 16 in an adjustable manner and is electrically connected to the controller 40 via a signal wire 17. The signal wire 17 passes through, but is not limited to, the auxiliary bar 16 and one of the temples as shown in FIG. 2. The electromagnetic actuator 31 can be mounted on the auxiliary bar 16 in a variety of ways; for example a carrier assembly that includes a base 32 and a rail 33 is used to hold the electromagnetic actuator 31. The rail 33 is provided on the top surface of the auxiliary bar 16. The base 32 for supporting the electromagnetic actuator 31 is engaged on and allowed to movably slide along the rail 33.

The controller 40 is an operable and portable device which can be worn on the clothes using a clip or placed in the pocket. The controller 40 is electrically connected to the biosignal sensor 20 and the electromagnetic actuator 31 via the signal wire 17 to receive and process the sensing data, and control the activation or deactivation of the electromagnetic actuator 31 based on the sensing data.

The release button 60 on the controller 40 is operable by the user. When the release button 60 is pressed continuously, the controller 40 disables the electromagnetic actuator 31 to release the magnetic element 50 from the electromagnetic actuator 31.

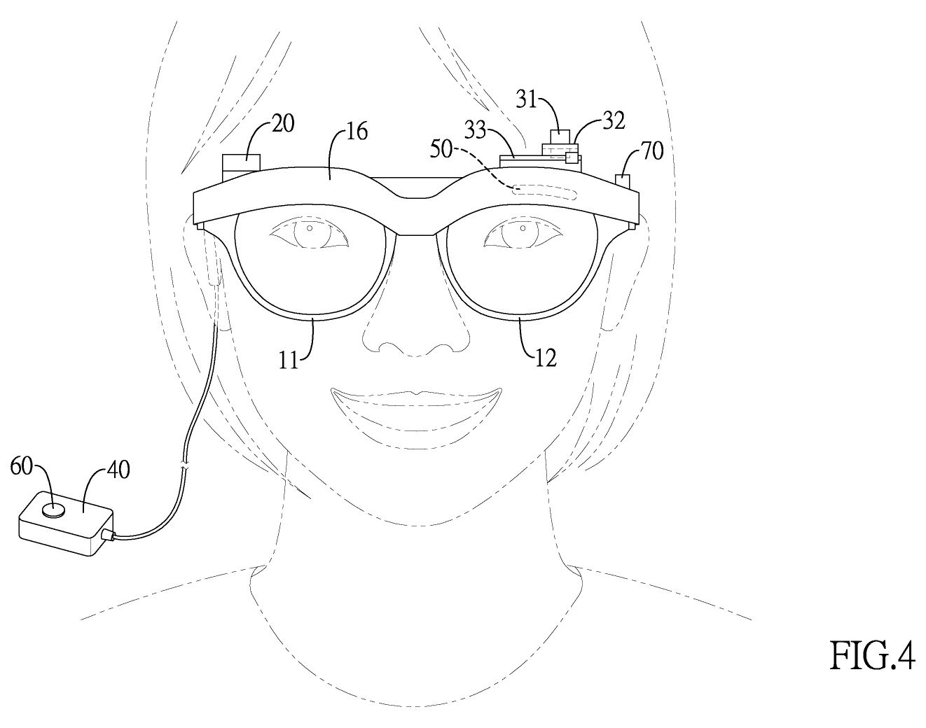

Referring to FIG. 4, the magnetic element 50 is configured to be affixed to the paralytic eyelid of the user. For example, the attachment of the magnetic element 50 to the paralytic eyelid can be achieved using a double-sided adhesive tape. By deactivating the electromagnetic actuator 31, the magnetic element 50 can be separated from the electromagnetic actuator 31. During the activation of the electromagnetic actuator 31, the magnetic element 50 is attracted upward by the produced magnetic force until the electromagnetic actuator 31 is deactivated. In another embodiment, the magnetic element 50 may be implanted in the paralytic eyelid. The magnetic element 50 may be a metal sheet, such as a magnetic sheet made of iron, nickel or cobalt.

Professionals such as physicians may operate the release button 60 to temporarily interrupt the magnetic attraction between the magnetic element 50 and the electromagnetic actuator 31, thereby adjusting the relative position of the electromagnetic actuator 31 in a manual way. By continuously pressing the release button 60, the operator is allowed to slide the electromagnetic actuator 31 along the rail 33, thereby adjusting its relative position without pulling the user’s eyelid.

In another circumstance, when the user experiences an accidental situation, such as a displacement of the wearable object 10 from the face, the user can press the release button 60 continuously to interrupt magnetic attraction between the magnetic element 50 and the electromagnetic actuator 31, thereby avoiding accidental injury.

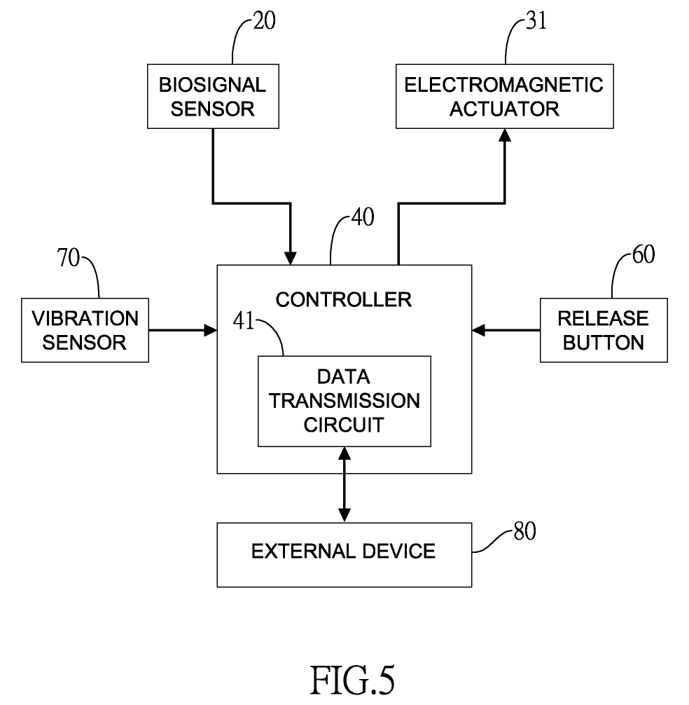

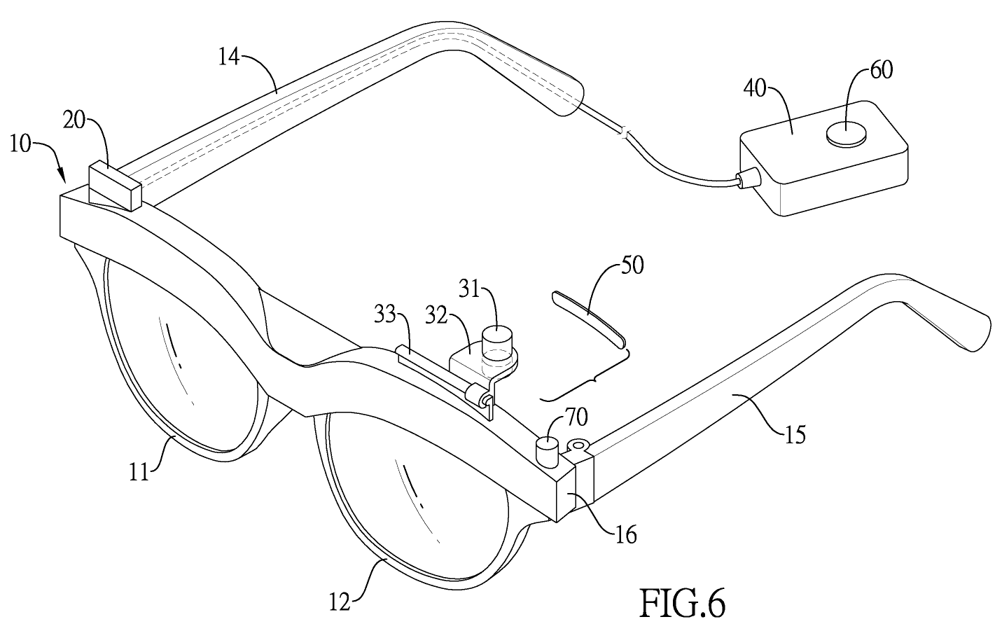

Further referring to FIGS. 5 and 6, another embodiment of the invention may further include one or more vibration sensors 70 disposed on the wearable object 10 and connected to the controller 40 electrically. The vibration sensor 70 is placed on the auxiliary bar 16 and corresponds to the paralytic eyelid. As an example, the vibration sensor 70 can be mounted on the top surface of the auxiliary bar 16 above the second lens frame 12 and near the second temple 15. Alternately, the vibration sensor 70 can be secured on the second temple 15.

The vibration sensor 70 such as an inertial sensor is configured to detect the vibration amplitude experienced by the wearable object 10. When the detected vibration amplitude exceeds a predetermined threshold, it typically indicates occurrence of an accidental event, such as a shaking, a collision, a fall, or an abrupt displacement of the wearable object 10. The vibration sensor 70 generates a vibration signal corresponding to the detected vibration amplitude and transmits it to the controller 40. By comparing the vibration sensing signal with the predetermined threshold, the controller 40 deactivates the electromagnetic actuator 31 to interrupt or terminate a magnetic attraction between the magnetic element 50 and the electromagnetic actuator 31 while the vibration sensing signal exceeds the preset threshold. Accordingly, upon the recognition of the accidental event such as the collision, the fall, or a sudden movement of the wearable object 10, the controller 40 disables the electromagnetic actuator 31, thereby separating the magnetic element 50 from the electromagnetic actuator 31 to enhance user safety and prevent potential injury.

### Application for blepharoptosis

According to one aspect of application, the magnetic assistance device of the present invention is designed for the blepharoptosis user. When the blepharoptosis user wears the magnetic assistance device as shown in FIG. 4, the electromagnetic actuator 31 is mounted above the second lens frame 12 at the side of the paralytic eyelid and near the eyebrow, and the magnetic element 50 is adhered to the paralytic eyelid. The blepharoptosis is a symptom present with the drooping of the upper eyelid. In order to assist the blepharoptosis users to open their eyes normally, the electromagnetic actuator 31 is activated most of the time, so that the electromagnetic actuator 31 attracts the magnetic element 50 upward to lift the drooping eyelid.

The biosignal sensor 20 detects the muscle or skin activities of the periorbital area around the healthy eyelid continuously and generates the sensing data. For example, after the near-infrared emitter continuously emits an infrared signal to the muscles around the healthy eyelid, the near-infrared receiver receives a reflected infrared signal from the healthy eyelid and accordingly produces the sensing data to the controller 40.

When the user closes the eyes, the muscle contractions around the eye will cause the variations in wavelength or potential of the reflected infrared light. Accordingly, the controller 40 can identify the eye closure of the healthy eyelid based on the reflected infrared light.

According to the recognition of eye closure, the controller 40 deactivates the electromagnetic actuator 31, thereby enabling the release of the magnetic element 50 from the electromagnetic actuator 31 and allowing the paralytic eyelid to close. After a predetermined eye-closure time, the controller 40 may activate the electromagnetic actuator 31 to re-attract the magnetic element 50. As a typical application, the electromagnetic actuator 31 is configured to attract the magnetic element 50, thus lifting and opening the paralytic eyelid. The user’s habit data such as the eye blinking frequency may be recorded simultaneously as a basis for setting the predetermined eye-closure time.

In another embodiment of this invention, the biosignal sensor 20 is a camera for continuously capturing image data of the healthy eye or the muscles around the healthy eyelid. The controller 40 may use a trained convolutional neural network (CNN) image recognizing model to process the image data, thereby identifying the eye open and closure to control the electromagnetic actuator 31 as described above.

### Application for lagophthalmos

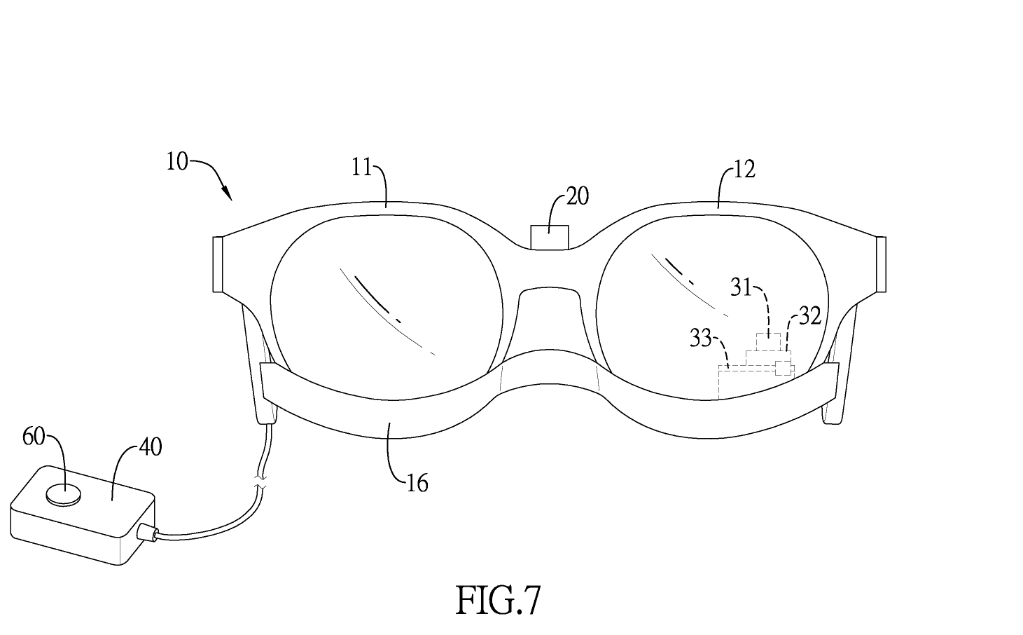

With reference to FIG. 7, in the fourth embodiment of the invention, the auxiliary bar 16 of the wearable object 10 is mounted to the lower rims instead of the upper rims of the first lens frame 11 and the second lens frame 12. The electromagnetic actuator 31 is disposed on the auxiliary bar 16 in correspondence with the second lens frame 12, and is positioned at an inner side of the second lens frame 12. In this embodiment, the magnetic assistance device is configured to assist a user exhibiting incomplete eyelid closure.

The electromagnetic actuator 31 may be mounted near the lower rim of the second lens frame 12 and close to the lower eye bag of the affected eyelid. The biosignal sensor 20 may be located in the bridge between the first lens frame 11 and the second lens frame 12 to detect muscle activities between the eyebrows. Alternately, the biosignal sensor 20 may be placed on the upper rim of the other lens frame in correspondence with the normal eyelid, and detect muscle contractions around the healthy upper eyelid during eye closures and openings. The magnetic element 50 is configured to be attached to the abnormal upper eyelid. For a user exhibiting incomplete eyelid closure, the electromagnetic actuator 31 is designed to attract the magnetic element 50 downward, thereby enabling the eyelid on the affected side to close completely.

As discussed above, the biosignal sensor 20 detects the skin of the periorbital area of the healthy eyelid or muscle activities between the eyebrows continuously, and generates the sensing data.

When the user open the eyes, the muscle contractions around the eye will cause the variations in wavelength or potential of the reflected infrared light. Accordingly, the controller 40 can identify the eye open of the healthy eyelid based on the reflected infrared light.

According to the recognition of eye open at the normal side, the controller 40 deactivates the electromagnetic actuator 31, thereby enabling the release of the magnetic element 50 from the electromagnetic actuator 31 and allowing the affected eyelid to open. In an embodiment, after a predetermined eye-open time, the controller 40 may activate the electromagnetic actuator 31 to re-attract the magnetic element 50 downward. As a typical application, the electromagnetic actuator 31 is configured to attract the magnetic element 50, thus closing the incomplete closure eyelid. The user’s habit data such as the eye blinking frequency may be recorded simultaneously as a basis for setting the predetermined eye-open time.

In another embodiment, the controller 40 further comprises a data transmission circuit 41, which transmits data such as blinking speed and frequency through a wireless or wired manner to an external device 80. The external device 80, for example a controller computer, can be operated by professionals such as physicians. The predetermined eye-closure time as mentioned is allowed to be adjusted through the external device 80.

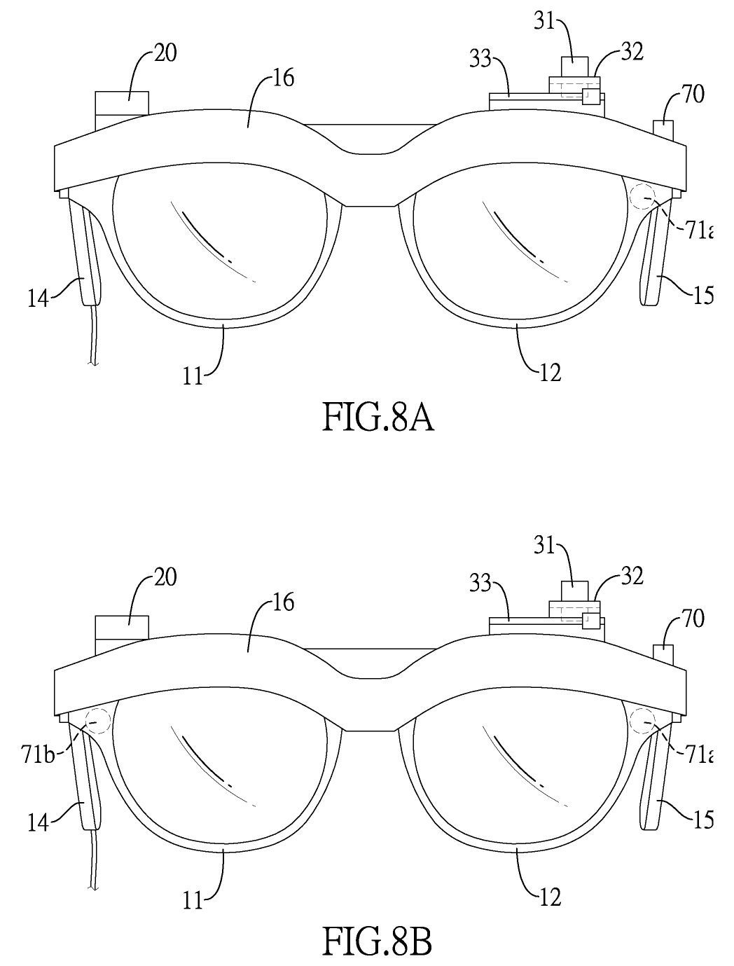

With reference to FIGS. 8A and 8B, according to other embodiments of the present invention, while the biosignal sensor 20 is an optical sensor, the magnetic assistance device may further include at least one camera 71a, 71b attached to the inner side of the wearable object 10. The camera 71a in FIG. 8A is preferably mounted on a corner of the second lens frame 12 near the second temple 15 for capturing images of the muscle activities or periorbital skin of the affected eyelid, or images of the eyeball. The image data captured by the camera 71a is transmitted to and processed by the controller 40. With the diversity of acquired data, including the sensing data from the optical sensor and the image data from the camera 71a, the controller 40 controls the electromagnetic actuator 31 more accurately.

In FIG. 8B, two cameras 71a and 71b are mounted on the inner sides of the first lens frame 11 and the second lens frame 12 respectively at symmetrical positions for capturing images of the muscle activities or periorbital skin of the eyelids or images of the eyeballs. By comparing the images data captured from eyes and evaluating their differences, the controller 40 determines whether the wearable object 10 is slipping from the face or subject to an accidental event such as a collision. For example, if the differences in size or position of the pupils images in the captured image data exceed a certain level, the controller 40 may determine the wearable object 10 has been shifted from the face, and accordingly deactivate the electromagnetic actuator 31 to release the magnetic element 50.

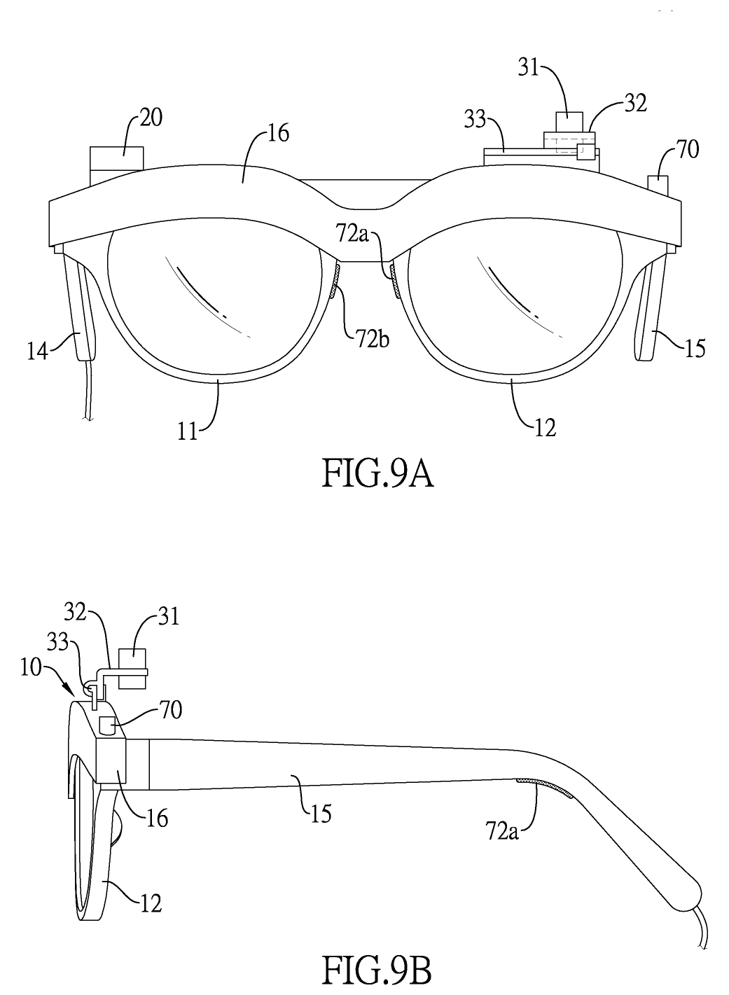

With reference to FIGS. 9A and 9B, according to other embodiments of the present invention, a pair of thin-film pressure sensors 72a, 72b is further added onto the wearable object 10. The two thin-film pressure sensors 72a, 72b are preferably configured to contact the user’s skin. For example, the two thin-film pressure sensors 72a, 72b can be affixed on nose pads opposite to each other of the glasses frame as shown in FIG. 9A. Alternatively, as shown in FIG. 9B, the two thin-film pressure sensors 72a, 72b may be affixed respectively at curved sections of the first temple 14 and the second temple 15 so that the thin-film pressure sensors 72a, 72b can contact the user’s skin approximately above the top of the ears. When the wearable object 10 is worn on the face well, pressure values sensed by the two thin-film pressure sensors 72a, 72b should be balanced to each other and approximately the same. Once the wearable object 10 slips off or is shifted from the face, an imbalance between the pressure values will cause a difference that exceeds a pressure threshold. When the displacement of the wearable object 10 is detected, the controller 40 immediately deactivates the electromagnetic actuator 31 to release the magnetic element 50, thereby preventing injury to the user’s eyelids.

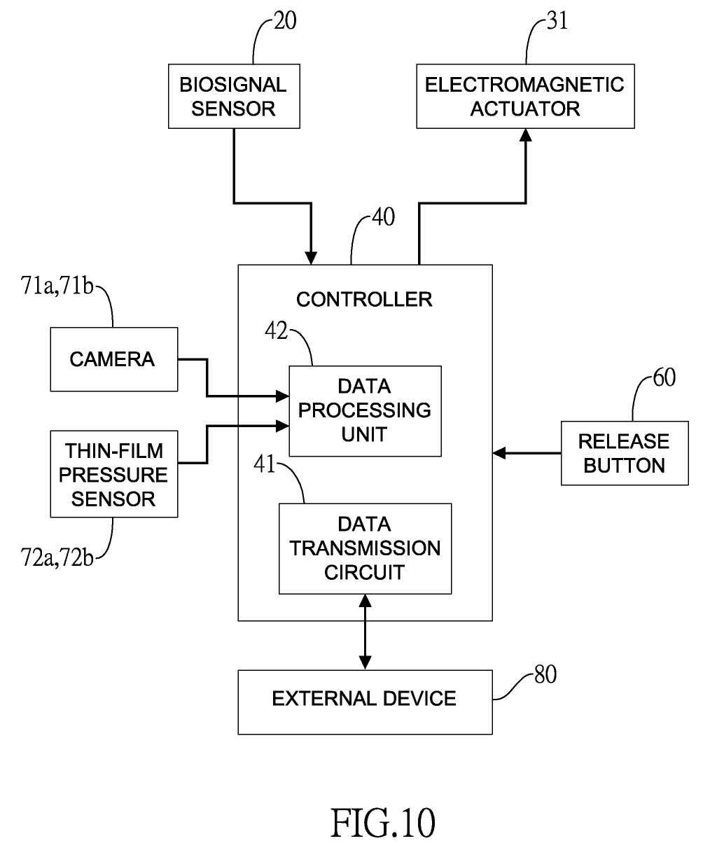

With reference to FIG. 10, for the foregoing magnetic assistant devices, the controller 40 has a data processing unit 42 for processing the captured image data and/or the sensed pressure values. The data processing unit 42 may use a trained image identifying model to correctly recognize the eyelid closure and open, and/or the displacement of the wearable object 10 from the user’s face.

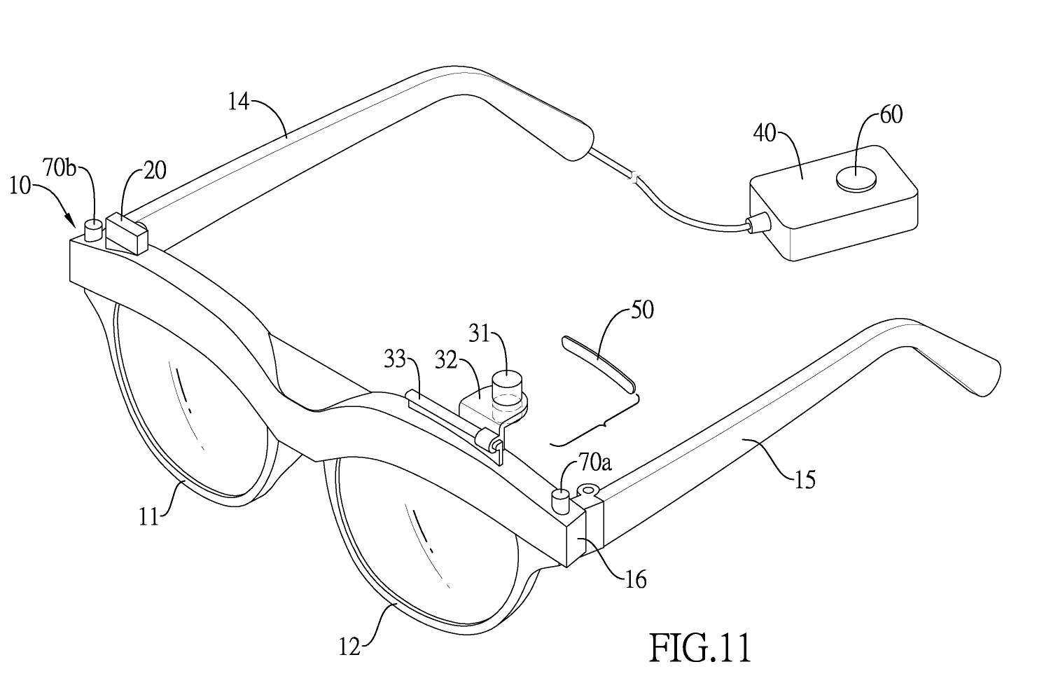

With reference to FIG. 11, according to another embodiment of the invention, two vibration sensors 70a, 70b are preferably mounted in a symmetrical distribution at opposite positions of the wearable object 10. Each vibration sensor 70a, 70b may be implemented by an inertial sensor for detecting the vibration amplitude experienced by the wearable object 10. Similar to the embodiments described above, each of the vibration sensors 70a, 70b generates a vibration signal corresponding to the detected vibration amplitude and transmits it to the controller 40. While any of the vibration sensing signals exceeds the predetermined vibration threshold, the controller 40 deactivates the electromagnetic actuator 31 to interrupt or terminate a magnetic attraction between the magnetic element 50 and the electromagnetic actuator 31. Accordingly, upon the recognition of the accident event, the controller 40 disables the electromagnetic actuator 31, thereby separating the magnetic element 50 from the electromagnetic actuator 31.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A feedback-enabled magnetic assistance device for eyelid management comprising:
a wearable object configured to be worn on a user’s face;
a magnetic element adapted to be affixed on an affected eyelid of the user;
a biosignal sensor mounted on the wearable object, detecting muscle activities in a periorbital area of the user, and producing sensing data corresponding to the muscle activities;
an electromagnetic actuator mounted on the wearable object at a position corresponding to the magnetic element, wherein the electromagnetic actuator is controllable to be activated to generate a magnetic attraction between the magnetic element and the electromagnetic actuator, or alternatively is controllable to be deactivated to interrupt the magnetic attraction; and
a controller electrically connected to the biosignal sensor and the electromagnetic actuator via a signal wire, and configured to activate or deactivate the electromagnetic actuator according to the sensing data acquired from the biosignal sensor;
a release button electrically configured on the controller, wherein the controller deactivates the electromagnetic actuator while the release button is triggered.

2. The device as claimed in claim 1, wherein at least one vibration sensor is mounted on the wearable object and electrically connected to the controller to detect a vibration amplitude experienced by the wearable object and to produce a vibration signal corresponding to the vibration amplitude; and
the controller receives the vibration signal and deactivates the electromagnetic actuator when the vibration signal exceeds a vibration threshold to release the magnetic element from the electromagnetic actuator.

3. The device as claimed in claim 2, wherein the biosignal sensor is an optical sensor composed of a near-infrared emitter and a near-infrared receiver.

4. The device as claimed in claim 2, wherein the biosignal sensor is a camera configured to capture image data of the user’s lateral canthus or images of eye closures and openings as the sensing data.

5. The device as claimed in claim 4, wherein the controller uses a convolutional neural network (CNN) image recognizing model to recognize the eye closures and openings in the image data.

6. The device as claimed in claim 2, wherein
the wearable object is an eyeglass frame and comprises:
a first lens frame;
a second lens frame;
a first temple pivotally connected to a side of the first lens frame;
a second temple pivotally connected to a side of the second lens frame; and
an auxiliary bar mounted on top rims of the first lens frame and the second lens frame;
the biosignal sensor is mounted on the auxiliary bar above the first lens frame;
the electromagnetic actuator and the vibration sensor are mounted on the auxiliary bar above the second lens frame; and
the magnetic element is an iron sheet.

7. The device as claimed in claim 6, wherein the controller electrically connects to the biosignal sensor and the electromagnetic actuator via the signal wire embedded in the first temple.

8. The device as claimed in claim 1, wherein
the electromagnetic actuator comprises an electromagnet and a permanent magnet;
the wearable object further comprises:
a rail provided on a top surface of the wearable object; and
a base movably disposed along the rail and carrying the electromagnetic actuator.

9. The device as claimed in claim 2, wherein
the wearable object is an eyeglass frame and comprises:
a first lens frame;
a second lens frame;
a first temple pivotally connected to the first lens frame;
a second temple pivotally connected to the second lens frame;
an auxiliary bar mounted on bottom rims of the first lens frame and the second lens frame; and
a bridge interconnected between the first lens frame and the second lens frame;
the biosignal sensor is mounted on the bridge;
the magnetic element is an iron sheet; and
the electromagnetic actuator and the vibration sensor are mounted on the auxiliary bar in correspondence with the iron sheet.

10. The device as claimed in claim 1, wherein the controller further comprises a data transmission circuit configured to communicate with an external device in a wired or wireless manner.

11. The device as claimed in claim 6, wherein the biosignal sensor is an optical sensor composed of a near-infrared emitter and a near-infrared receiver, and the wearable object further comprises a camera mounted at an inner side of the second lens frame and electrically connected to the controller.

12. The device as claimed in claim 6, wherein
the biosignal sensor is an optical sensor composed of a near-infrared emitter and a near-infrared receiver;
the wearable object further comprises:
two cameras mounted on inner sides of the first lens frame and the second lens frame respectively and symmetrically, and electrically connected to the controller, wherein the two cameras capture image data of periorbital skin of the eyelids or image data of the eyeballs; and
the controller deactivates the electromagnetic actuator when the image data captured by the two cameras meet a condition.

13. The device as claimed in claim 6, wherein
two thin-film pressure sensors are mounted symmetrically on the wearable object for contacting the user’s skin, and each of the thin-film pressure sensors is electrically connected to the controller and senses a pressure value;
the controller deactivates the electromagnetic actuator when a difference between the pressure values sensed by the two thin-film pressure sensors exceeds a pressure threshold.

14. The device as claimed in claim 13, wherein the eyeglass frame further comprises two nose pads; and the two thin-film pressure sensors are mounted on the two nose pads respectively.

15. The device as claimed in claim 13, wherein the two thin-film pressure sensors are affixed at curved sections of the first temple and the second temple respectively.

16. The device as claimed in claim 6, wherein the at least one vibration sensor comprises two inertial sensors mounted at opposite positions symmetrically on the wearable object;
each of the inertial sensors detects the vibration amplitude experienced by the wearable object, and generates the vibration signal corresponding to the vibration amplitude detected; and
the controller deactivates the electromagnetic actuator when any of the vibration signals exceeds the vibration threshold.
